(19) United States
(12) Patent Application Publication
(10) Pub. No.: US 2024/0408112 A1
(43) Pub. Date: Dec. 12, 2024

(54) METHOD FOR PREVENTING OR TREATING PULMONARY ARTERIAL HYPERTENSION BY USING 7-DEHYDROCHOLESTEROLS OR SALTS, OXIDES, METABOLITES, OR DERIVATIVES THEREOF

(71) Applicant: EXCELSIOR PHARMATECH LABS., Taipei City (TW)

(72) Inventor: Joseph Chen, Taipei City (TW)

(73) Assignee: EXCELSIOR PHARMATECH LABS., Taipei City (TW)

(21) Appl. No.: 18/734,287

(22) Filed: Jun. 5, 2024

Related U.S. Application Data
(60) Provisional application No. 63/471,322, filed on Jun. 6, 2023.

(51) Int. Cl. A61K 31/58; A61K 45/06

(52) U.S. Cl. CPC A61K 31/58; A61K 45/06

(57) ABSTRACT

Provided is a method for preventing or treating pulmonary arterial hypertension in a subject in need thereof, including administering to the subject an effective amount of a 7-dehydrocholesterols or a salt, an oxide, a metabolite, or a derivative thereof.

### REFERENCE TO RELATED APPLICATIONS

This application claims priority to, and the benefit of the filing date of U.S. Provisional Application No. 63/471,322, filed on Jun. 6, 2023, the entire contents of which, including all drawings thereof, in any language, are incorporated herein by reference.

### BACKGROUND

### 1. Technical Field

The present disclosure relates to a method for preventing or treating pulmonary arterial hypertension, particularly a method for preventing or treating pulmonary arterial hypertension by using 7-dehydrocholesterol or a salt, an oxide, a metabolite or a derivative thereof.

### 2. Description of Related Art

Pulmonary arterial hypertension (PAH) is a destructive pulmonary vascular disease characterized by persistently elevated mean arterial pressure in pulmonary vessels (>25 mmHg at rest) and normal pulmonary capillary wedge pressure of no more than 15 mmHg accompanying by increase of vessels, thereby progressively leading to right heart failure and even death^{1,2}. Pulmonary arterial hypertension may be primary, hereditary, drug- or toxin-induced, or associated with other conditions including connective tissue diseases, HIV infection, portal hypertension, congenital heart disease, and schistosomiasis, etc.³ However, the pathogenesis of pulmonary arterial hypertension remains unclear until now.

Early pathological changes of tissues of pulmonary arterial hypertension include vascular endothelial fibrosis, smooth muscle cell proliferation and peripheral pulmonary artery obstruction⁴. Plexiform lesions are pathological changes in late-stage severe pulmonary arterial hypertension, which originate from the proliferation of pulmonary artery endothelial cells, smooth muscle cells, and circulating cells^{5,6}. Relevant genetic studies show that bone morphogenetic protein receptor type 2 (BMPR2) is an important gene affecting more than 70% of patients with familial pulmonary arterial hypertension (FPAH) and 10 to 20% of patients with idiopathic pulmonary arterial hypertension (HPAH)⁷⁻¹⁰. New classification system reclassifies these patients as hereditable pulmonary arterial hypertension (HPAH).

BMPR2 is a member of the transforming growth factor-β (TGF-β) receptor superfamily. TGF-β is a multifunctional cytokine that is related to cell growth, differentiation, apoptosis, angiogenesis, wound healing, neuroprotection and immune regulation. Pathologies related to TGF-β include immunosuppression, inflammation, vascular sclerosis, neurodegeneration, tissue fibrosis, and cancer, etc., and promoting or inhibiting TGF-β has become the target for developing many new drugs.

7-dehydrocholesterol (7-DHC) is a TGF-β receptor inhibitor, as disclosed in U.S. Pat. No. 8,946,201, a certain amount of oxidized 7-DHC can effectively inhibit the activity of TGF-β in the subject, thereby treating and/or preventing skin diseases such as skin fibrosis, skin wounds, inflammation, and alopecia. PCT Patent Publication No. WO2009/138582 also indicates that compositions containing 7-DHC, its derivatives or natural extracts of plant or animal microorganisms containing 7-DHC can be used as cosmetics or food additives. U.S. Pat. No. 10,683,324 mentions that 7-DHC can be used to treat or prevent cancer, as well as treat or prevent uncontrolled angiogenesis.

The drugs traditionally used to treat pulmonary arterial hypertension include calcium ion blockers, anticoagulants, diuretics or cardiotonic agents. In recent years, some new drugs have been developed for the treatment of pulmonary arterial hypertension, which can be divided into three categories according to the mechanism of action: (1) endothelin receptor antagonists; (2) phosphodiesterase type 5 inhibitors; and (3) prostacyclin analogs. However, both traditional drugs and novel drugs can only slow down the progression of the disease and temporarily improve the clinical symptoms of the patient.

Therefore, there is still an urgent need to develop a method or medicine effective for preventing or treating pulmonary arterial hypertension.

### SUMMARY

The present disclosure provides a method for preventing or treating pulmonary arterial hypertension using a pharmaceutical composition comprising a TGF-β receptor inhibitor and a pharmaceutically acceptable excipient thereof.

In one aspect, in view of the foregoing, the present disclosure provides a pharmaceutical composition for preventing or treating pulmonary arterial hypertension in a subject in need thereof, comprising an effective amount of a TGF-β receptor inhibitor and a pharmaceutically acceptable excipient thereof.

In at least one embodiment of the present disclosure, the TGF-β receptor inhibitor is a 7-dehydrocholesterol or a salt, an oxide, a metabolite or a derivative thereof.

In at least one embodiment of the present disclosure, the 7-dehydrocholesterol, or a salt, an oxide, a metabolite or a derivative thereof is administered to the subject in a dosage range of 1 mg/kg to 50 mg/kg.

In some embodiments of the present disclosure, the 7-dehydrocholesterol, or a salt, an oxide, a metabolite or a derivative thereof is administered to the subject in a dosage range of 10 mg/kg to 20 mg/kg.

In at least one embodiment of the present disclosure, the 7-dehydrocholesterol, or a salt, an oxide, a metabolite or a derivative thereof is a single active ingredient in the pharmaceutical composition. In other embodiments of the present disclosure, the pharmaceutical composition further comprises an additional active ingredient selected from the group consisting of a calcium blocker, an anticoagulant, a diuretic, a cardiotonic agent, an endothelin receptor antagonist, a phosphodiesterase type 5 inhibitor, a prostacyclin analog, and any combination thereof.

In another aspect, in view of the foregoing, the present disclosure provides a method for preventing or treating pulmonary arterial hypertension in a subject in need thereof, comprising administering to the subject a pharmaceutical composition comprising an effective amount of a TGF-β receptor inhibitor and a pharmaceutically acceptable excipient thereof.

In at least one embodiment of the present disclosure, the TGF-β receptor inhibitor can reduce proliferation of pulmonary artery endothelial cells, smooth muscle cells, or a combination thereof in the subject.

In at least one embodiment of the present disclosure, the TGF-β receptor inhibitor is a 7-dehydrocholesterol, or a salt, an oxide, a metabolite or a derivative thereof.

In at least one embodiment of the present disclosure, the salt of 7-dehydrocholesterol includes an acetate salt or a benzoate salt of a (3β)-7-dehydrocholesterol or a derivative thereof.

In at least one embodiment of the present disclosure, the derivative of 7-dehydrocholesterol includes a cholecalciferol (i.e., vitamin D3) or a compound represented by formula (I), in which R₁ is CR₅ or N; R₃ is selected from the group consisting of —O(CR₅)ₙR₆, —OC(—O)(CR₅)ₙR₆, —OC(═O)(CR₅)ₙOR₅ and —OC(═O)C(R₅)═C(R₅)₂; R₂ is selected from the group consisting of oxygen, sulfur, C(R₄)₂, and N(R₄); R₄ is independently selected for each occurrence from the group consisting of hydrogen, alkyl, substituted alkyl, cycloalkyl, substituted cycloalkyl, alkenyl, substituted alkenyl, alkynyl, substituted alkynyl, aryl, substituted aryl, aralkyl, substituted aralkyl, heteroaryl, substituted heteroaryl, heteroarylalkyl, substituted heteroarylalkyl, OR₅ and N(R₅)₂; R₅ is independently selected for each occurrence from the group consisting of hydrogen, alkyl, substituted alkyl, cycloalkyl, substituted cycloalkyl, alkenyl, substituted alkenyl, alkynyl, substituted alkynyl, aryl, substituted aryl, aralkyl, substituted aralkyl, heteroaryl, substituted heteroaryl, heteroarylalkyl and substituted heteroarylalkyl; R₆ is selected from the group consisting of fluorine, chlorine, bromine, iodine, methanesulfonyl, toluenesulfonyl, —OSi(R₅)₃, —C(═O)OR₅ and —C(═O)R₅; dashed line represents a single bond or a double bond; and n is an integer from 1 to 10.

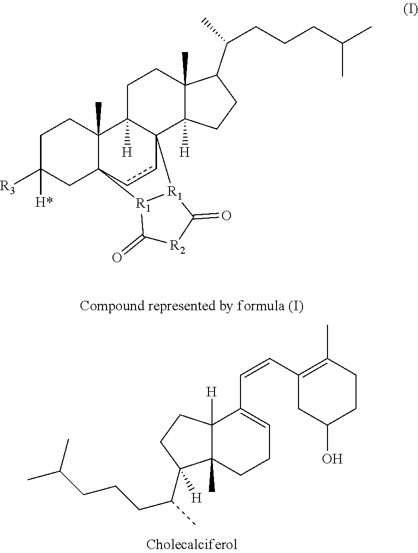

In at least one embodiment of the present disclosure, the compound represented by formula (I) is the compound represented by formula (Ia) or a salt or a solvate thereof, in which R₁ to R₃ are as defined above:

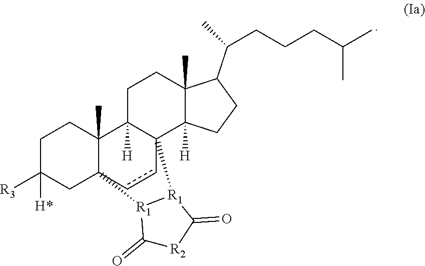

In at least one embodiment of the present disclosure, the compound represented by formula (I) is the compound represented by formula (Tb) or a salt or a solvate thereof, wherein R₁ to R₃ are as defined above:

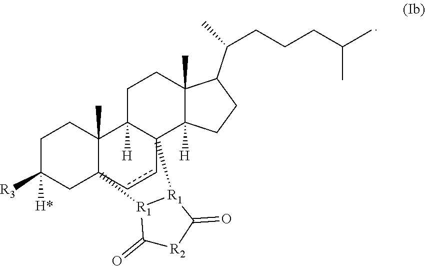

In at least one embodiment of the present disclosure, R₁ of the compound represented by formula (I) is N.

In at least one embodiment of the present disclosure, R₂ is N(R₄), and R₄ is defined as above.

In at least one embodiment of the present disclosure, the compound represented by formula (I) is the compound represented by formula (Ic) or a salt or a solvate thereof, in which R₃ and R₄ are as defined above:

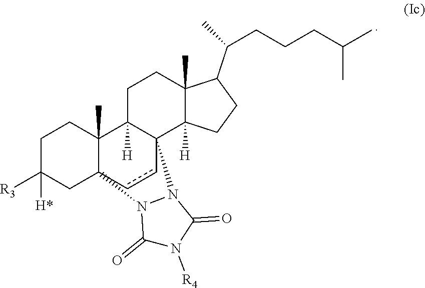

In at least one embodiment of the present disclosure, the compound represented by formula (I) is the compound represented by formula (Id) or a salt or a solvate thereof, in which R₃ and R₄ are as defined above:

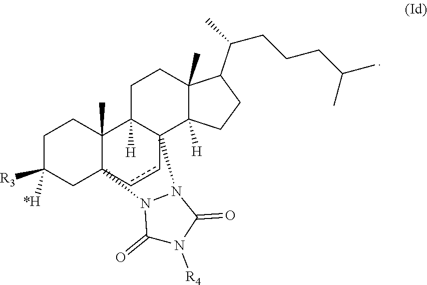

In at least one embodiment of the present disclosure, the compound represented by formula (I) is

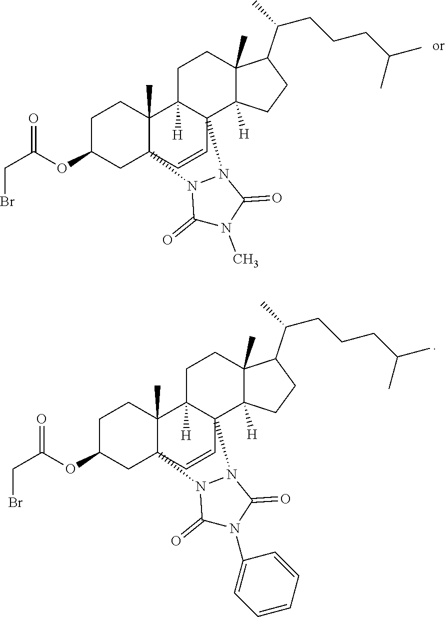

In at least one embodiment of the present disclosure, the subject is a mammal.

In at least one embodiment of the present disclosure, the subject is human.

In at least one embodiment of the present disclosure, the compound represented by formula (I) is

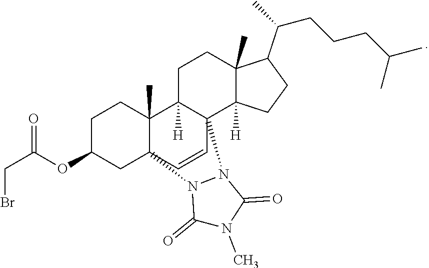

In at least one embodiment of the present disclosure, the 7-dehydrocholesterol, or a salt, an oxide, a metabolite or a derivative thereof is administered to the subject at a dose ranging from 1 mg/kg to 50 mg/kg.

In at least one embodiment of the present disclosure, the 7-dehydrocholesterol, or a salt, an oxide, a metabolite or a derivative thereof is administered to the subject at a dose ranging from 10 mg/kg to 20 mg/kg.

In at least one embodiment of the present disclosure, the TGF-β receptor inhibitor can be administered to the subject in combination with an additional active ingredient to prevent or treat pulmonary arterial hypertension.

In at least one embodiment of the present disclosure, the 7-dehydrocholesterol, or a salt, an oxide, a metabolite or a derivative is a single active ingredient in the pharmaceutical composition for preventing or treating the pulmonary arterial hypertension.

In other embodiments of the present disclosure, the additional active ingredient is selected from the group consisting of a calcium blocker, an anticoagulant, a diuretic, a cardiotonic agent, an endothelin receptor antagonist, a phosphodiesterase type 5 inhibitor, a prostacyclin analog, and any combination thereof.

In other embodiments of the present disclosure, the TGF-β receptor inhibitor reduces the proliferation of pulmonary artery endothelial cells, smooth muscle cells, or a combination thereof in the subject.

In other embodiments of the present disclosure, the pharmaceutically acceptable excipient includes a filler, a binder, a preservative, a disintegrant, a lubricant, a suspending agent, a wetting agent, a solvent, a surfactant, an acid, a flavoring agent, polyethylene glycol, alkanes glycol, sebacic acid, dimethyl sulfoxide, alcohol or any combination thereof.

In other embodiments of the present disclosure, the pharmaceutical composition is a formulation selected from the group consisting of troches, tablets, liquids, powders, granules, dispersants, pills, dripping pills, capsules, ointments, creams, emulsions, gels, patches, injections, inhalants, sprays and suppositories.

In other embodiments of the present disclosure, the pharmaceutical composition is subcutaneously, intravenously, intradermally, intraperitoneally, orally, intrabuccally, sublingually, or intramuscularly, or through respiratory tract, or lungs administered to the subject.

In other embodiments of the present disclosure, the pharmaceutical composition is administered to the subject at least once daily.

In other embodiments of the present disclosure, the pharmaceutical composition is administered to the subject for a period of at least 1 month.

In addition to the above, the present disclosure further provides a use of a pharmaceutical composition comprising an effective amount of 7-dehydrocholesterol, or a salt, an oxide, a metabolite or a derivative thereof in the manufacture of a medicament for preventing or treating pulmonary arterial hypertension in a subject in need thereof. Moreover, the present disclosure further provides the use of an effective amount of 7-dehydrocholesterol, or a salt, an oxide, a metabolite or a derivative thereof in the manufacture of a medicament for preventing or treating pulmonary arterial hypertension in a subject in need thereof.

Also, the present disclosure provides a pharmaceutical composition comprising an effective amount of 7-dehydrocholesterol, or a salt, an oxide, a metabolite or a derivative thereof for use in preventing or treating pulmonary arterial hypertension in a subject in need thereof and an effective amount of 7-dehydrocholesterol, or a salt, an oxide, a metabolite or a derivative thereof for use in preventing or treating pulmonary arterial hypertension in a subject in need thereof.

### BRIEF DESCRIPTIONS OF THE DRAWINGS

The present disclosure can be more fully understood by reading the following description of the embodiments, with reference made to the accompanying drawings.

FIG. 1 shows the cell survival assay (MTT assay) results of 7-DHC (039) in normal rat pulmonary artery smooth muscle cells (CON1), PAH rat pulmonary artery smooth muscle cells (rPASMC MCT4), human pulmonary artery endothelial cells (hPAEC) and human pulmonary artery smooth muscle cells (hPASMC).

FIG. 2 shows the conditions of the cell growth inhibitions of BrdU cell proliferation assay by 7-DHC (039) for normal rat pulmonary artery smooth muscle cells (rPASMC CON1), PAH rat pulmonary artery smooth muscle cells (rPASMC MCT4), human pulmonary artery endothelial cells (hPAEC) and human pulmonary artery smooth muscle cells (hPASMC) under the conditions of 10% FBS.

FIG. 3 shows the conditions of the cell growth inhibitions of BrdU cell proliferation assay by 7-DHC (039) for normal rat pulmonary artery smooth muscle cells (rPASMC CON3), PAH rat pulmonary artery smooth muscle cells (rPASMC MCT2), human pulmonary artery endothelial cells (hPAEC) and human pulmonary artery smooth muscle cells (hPASMC) under the condition of 0.1% FBS, 100 pM TGF-β.

FIG. 4 illustrates the administration schedule, animal survival status and body weight (BW) changes of the pulmonary hypertension animal model experiment of a control group (CON), MCT induced pulmonary hypertension rat group (MCT), 10 mg/kg 7-DHC treated pulmonary hypertension rat group (039 10 mg/kg), 20 mg/kg 7-DHC treated pulmonary hypertension rat group (039 20 mg/kg) and 50 mg/kg 042 compound treated pulmonary hypertension rat group (042 50 mg/kg). Graphs indicated by slashes represent dead rats in different groups.

FIG. 5 shows the physiological parameters of the rats tested after administration in the pulmonary arterial hypertension animal model, including right ventricle pressure (RVP), systemic mean blood pressure (mbp), heart rate (HR) and right ventricle hypertrophy (RV/S+LV; right ventricle (RV)/septum (S)+left ventricle (LV)).

FIG. 6 is immunohistological staining diagrams of the pulmonary artery vascular tissue section of the rats tested in the pulmonary arterial hypertension animal model, and a histogram of the pulmonary artery blood vessel thickening degree of rats in different administration groups, and the different administration groups include a control group (CON), pulmonary hypertension rat group (MCT), 10 mg/kg 7-DHC treated pulmonary hypertension rat group (expressed as “039 10 mg”), 20 mg/kg 7-DHC treated pulmonary hypertension rat group (expressed as “039 20 mg”), and 50 mg/kg 042 compound treated pulmonary hypertension rat group (expressed as “042 50 mg”).

FIG. 7 shows the arterial gas analysis values of the rats tested after administration in the pulmonary hypertension animal model, including blood pH, oxygen pressure (PaO₂), carbon dioxide pressure (PaCO₂), total amount of carbon dioxide (TCO₂) in the blood of three forms (CO₂, H₂CO₃, and dissociated HCO₃⁻ ions), bicarbonate (HCO₃) in the form of dissociated HCO₃⁻ ions and base excess in the extracellular fluid compartment (BEecf).

FIG. 8 illustrates the administration schedule, animal survival status and body weight (BW) changes of the pulmonary hypertension animal model experiments of a control group (CON), a pulmonary hypertension rat (MCT), 10 mg/kg 7-DHC treated pulmonary hypertension rat group (expressed as “039 10 mg/kg”), 20 mg/kg 7-DHC treated pulmonary hypertension rat group (expressed as “039 20 mg/kg”) and 30 mg/kg macitentan treated pulmonary hypertension rat group (expressed as “macitentan 30 mg/kg”). Graphs indicated by slashes represent dead rats in different groups.

FIG. 9 shows the physiological parameters of the rats tested after administration in the pulmonary arterial hypertension animal model, including right ventricle pressure (RVP), systemic mean blood pressure (mbp), heart rate (HR) and right ventricle hypertrophy (RV/S+LV).

FIG. 10 is immunohistological staining diagrams of the pulmonary artery vascular tissue section of the rats tested in the pulmonary arterial hypertension animal model, and a histogram of the pulmonary artery blood vessel thickening degree of rats in different administration groups (statistical blood vessel wall thickness for blood vessels with a diameter between 50-100 m), and the different administration groups include a control group (CON), pulmonary hypertension rat group (MCT), 10 mg/kg 7-DHC treated pulmonary hypertension rat group (expressed as “039 10 mg”), 20 mg/kg 7-DHC treated pulmonary hypertension rat group (expressed as “039 20 mg”), and 30 mg/kg macitentan treated pulmonary hypertension rat group (expressed as “macitentan 30 mg”).

FIG. 11 shows the arterial gas analysis values of the rats tested after administration in the pulmonary hypertension animal model, including carbon dioxide pressure (PaCO₂), oxygen pressure (PaO₂), total amount of carbon dioxide (TCO₂), oxygen saturation (SO₂), base excess in the extracellular fluid compartment (BEecf) and bicarbonate (HCO₃). The normal value of SO₂ is 93% to 100%. Under a certain PaO₂, the percentage of oxygenated Hb in the blood sample to the total Hb reflects the degree of combination of O₂ and Hb in the blood, but is less sensitive to hypoxia than PaO₂.

FIG. 12A shows the conditions of inhibition of Pai-1 luciferase activity with or without TGF-β stimulation by 039 compound in different concentrations (0, 0.5, 1, 10, 25, 50 and 75 μM).

FIG. 12B shows the conditions of inhibition of TGF-β-stimulated Pai-1 luciferase activity (%) by 039 compound in different concentration ranges (0 to 80 μM).

FIG. 13A shows the conditions of inhibition of Pai-1 luciferase activity with or without TGF-β stimulation by 042 compound in different concentrations (0, 0.01, 0.1, 1, 5, 10 and 25 μM).

FIG. 13B shows the conditions of inhibition of TGF-β-stimulated Pai-1 luciferase activity (%) by 042 compound in different concentration ranges (0 to 30 μM).

FIG. 14A shows the conditions of inhibition of Pai-1 luciferase activity with or without TGF-β stimulation by 050 compound in different concentrations (0, 1, 5, 10, 25, 50 and 100 μM).

FIG. 14B shows the conditions of inhibition of TGF-β-stimulated Pai-1 luciferase activity (%) by 050 compound in different concentration ranges (0 to 120 μM).

FIG. 15A shows the conditions of inhibition of Pai-1 luciferase activity with or without TGF-β stimulation by 053 compound in different concentrations (0, 50, 75, 100, 150 and 200 μM).

FIG. 15B shows the conditions of inhibition of TGF-β-stimulated Pai-1 luciferase activity (%) by 053 compound in different concentration ranges (0 to 200 μM).

FIG. 16A shows the conditions of inhibition of Pai-1 luciferase activity with or without TGF-β stimulation by 056 compound in different concentrations (0, 0.1, 1, 10, 50 and 100 μM).

FIG. 16B shows the conditions of inhibition of TGF-β-stimulated Pai-1 luciferase activity (%) by 056 compound in different concentration ranges (0 to 120 μM).

FIG. 17A shows the conditions of inhibition of Pai-1 luciferase activity with or without TGF-β stimulation by 057 compound in different concentrations (0, 0.01, 0.1, 1, 10 and 100 μM).

FIG. 17B shows the conditions of inhibition of TGF-β-stimulated Pai-1 luciferase activity (%) by 057 compound in different concentration ranges (0 to 120 μM).

FIG. 18A shows the conditions of inhibition of Pai-1 luciferase activity with or without TGF-β stimulation by 203 compound in different concentrations (0, 5, 10, 25, 50 and 75 μM).

FIG. 18B shows the conditions of inhibition of TGF-β-stimulated Pai-1 luciferase activity (%) by 203 compound in different concentration ranges (0 to 70 μM).

### DETAILED DESCRIPTIONS

The following examples are used to illustrate the present disclosure. A person skilled in the art can easily conceive of the other advantages and effects of the present disclosure based on the invention of the specification. The present disclosure can also be implemented or applied as described in various examples. It is possible to modify or alter the following examples for carrying out the present disclosure without violating its spirit and scope, for different aspects and applications.

It should also be noted that the singular forms “a,” “an,” and “the” as used in this disclosure include plural referents unless expressly and unambiguously limited to one referent. In addition, the term “or” is used interchangeably with the term “and/or” unless the context clearly dictates otherwise.

As used herein, the term “comprising,” “having,” “including” or “containing” is used in reference to compositions, methods, and respective component(s) thereof, which are essential to the present disclosure, yet open to the inclusion of unspecified elements, whether essential or not. Additionally, the compositions of the present disclosure can be used to implement the methods of the present disclosure.

As used herein, the term “treating” or “treatment” refers to the administration of an effective amount of a 7-DHC or a salt or a derivative thereof to a subject in need thereof for curing, mitigating, relieving, remedying, ameliorating or preventing a disease its symptoms or its predisposition. This subject can be identified by a healthcare professional based on results from any suitable diagnostic method.

The present disclosure provides a method of treating pulmonary arterial hypertension in a subject in need thereof, comprising administering to the subject an effective amount of a 7-DHC or a salt or a derivative thereof.

As used herein, the term “an effective amount” refers to a therapeutic amount sufficient to result in the prevention or treatment of pulmonary arterial hypertension and the development, recurrence or onset of one or more symptoms thereof, or a therapeutic amount sufficient to enhance or improve the preventive effect of another therapy, reduce the severity and duration of a condition, improve one or more symptoms of a condition, prevent the progression of pulmonary arterial hypertension, and/or enhance or improve the therapeutic effect of another therapy.

The term “alkyl group” means a straight or branched saturated monovalent hydrocarbon chain having 1 to 12 carbon atoms. The straight chain or branched chain alkyl group having 1 to 6 carbon atoms is preferable. Examples thereof are methyl group, ethyl group, propyl group, isopropyl group, butyl group, t-butyl group, isobutyl group, pentyl group, hexyl group, isohexyl group, heptyl group, 4,4-dimethylpentyl group, octyl group, 2,2,4-trimethylpentyl group, nonyl group, decyl group, and various branched chain isomers thereof. Further, the alkyl group may optionally and independently be substituted by 1 to 4 substituents as listed below, if necessary.

The term “cycloalkyl group” means a monocyclic or bicyclic monovalent saturated hydrocarbon ring having 3 to 12 carbon atoms, and the monocyclic saturated hydrocarbon group having 3 to 7 carbon atoms is more preferable. Examples thereof are a monocyclic alkyl group and a bicyclic alkyl group such as cyclopropyl group, cyclobutyl group, cyclopentyl group, cyclohexyl group, cycloheptyl group, cyclooctyl group, cyclodecyl group, etc. These groups may optionally and independently be substituted by 1 to 4 substituents as mentioned below, if necessary.

The term “alkenyl group” means a straight or branched monovalent hydrocarbon chain having 2 to 12 carbon atoms and having at least one double bond. Preferable alkenyl group is a straight chain or branched chain alkenyl group having 1 to 6 carbon atoms. Examples thereof are vinyl group, 2-propenyl group, 3-butenyl group, 2-butenyl group, 4-pentenyl group, 3-pentenyl group, 2-hexenyl group, 3-hexenyl group, 2-heptenyl group, 3-heptenyl group, 4-heptenyl group, 3-octenyl group, 3-nonenyl group, 4-decenyl group, 3-undecenyl group, 4-dodecenyl group, 4,8,12-tetradecatrienyl group, etc. The alkenyl group may optionally and independently be substituted by 1 to 4 substituents as mentioned below, if necessary.

The term “alkynyl group” means a straight or branched monovalent hydrocarbon chain having at least one triple bond. The preferable alkynyl group is a straight chain or branched chain alkynyl group having 1 to 6 carbon atoms. Examples thereof are 2-propynyl group, 3-butynyl group, 2-butynyl group, 4-pentynyl group, 3-pentynyl group, 2-hexynyl group, 3-hexynyl group, 2-heptynyl group, 3-heptynyl group, 4-heptynyl group, 3-octynyl group, 3-nonynyl group, 4-decynyl group, 3-undecynyl group, 4-dodecynyl group, etc. The alkynyl group may optionally and independently be substituted by 1 to 4 substituents as mentioned below, if necessary.

The term “aryl group” means a monocyclic or bicyclic monovalent aromatic hydrocarbon group having 6 to 10 carbon atoms. Examples thereof are phenyl group, naphthyl group (including 1-naphthyl group and 2-naphthyl group). These groups may optionally and independently be substituted by 1 to 4 substituents as mentioned below, if necessary.

The term “aralkyl” as used alone or as part of another group refers to alkyl groups as described above having an aryl substituent. The aralkyl group may optionally and independently be substituted by 1 to 4 substituents as mentioned below, if necessary.

The term “heteroaryl” means a monocyclic or bicyclic monovalent aromatic hydrocarbon group having 6 to 10 carbon atoms, in which at least one carbon atom is substituted by at least one heteroatom, such as N, O or S. The heteroaryl group may optionally and independently be substituted by 1 to 4 substituents as mentioned below, if necessary.

The terms “heteroaralkyl” as used alone or as part of another group refers to alkyl groups as described above having an heteroaryl substituent. The heteroaralkyl group may optionally and independently be substituted by 1 to 4 substituents as mentioned below, if necessary.

The substituent for the above groups includes, for example, a halogen atom (e.g., fluorine, chlorine, bromine, iodine), a nitro group, a cyano group, an oxo group, a hydroxy group, a mercapto group, a carboxyl group, a sulfo group, an alkyl group, an alkenyl group, an alkynyl group, a cycloalkyl group, a cycloalkenyl group, a cycloalkynyl group, an aryl group, a heterocyclyl group, an alkoxy group, and a cycloalkyloxy group, but is not limited thereto.

In some embodiments of the present disclosure, the effective amount of 7-DHC or a salt and a derivative thereof may be 1 mg/kg to 50 mg/kg. In some embodiments, the lower limit of the dose may be 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg or 30 mg/kg, and the upper limit of the dose may be 50 mg/kg, 48 mg/kg, 45 mg/kg, 43 mg/kg, 40 mg/kg, 38 mg/kg, 35 mg/kg, 33 mg/kg, 30 mg/kg, 25 mg/kg, 24 mg/kg, 23 mg/kg, 22 mg/kg, 21 mg/kg or 20 mg/kg. For example, the dosage of the 7-DHC or a salt or a derivative thereof may be 1 mg/kg to 40 mg/kg, 5 mg/kg to 40 mg/kg, 5 mg/kg to 35 mg/kg, 10 mg/kg to 30 mg/kg, 10 mg/kg to 20 mg/kg, about 42 mg/kg, about 40 mg/kg, about 37 mg/kg, about 35 mg/kg, about 32 mg/kg, about 30 mg/kg, about 28 mg/kg, about 25 mg/kg, about 20 mg/kg, about 18 mg/kg, about 15 mg/kg, about 13 mg/kg or about 10 mg/kg.

As used herein, when referring to a number or range, a person having ordinary skill in the art can understand that it is intended to encompass an appropriate and reasonable range for the particular field to which the present disclosure relates.

In some embodiments of the present disclosure, the administration of 7-DHC or a salt or a derivative thereof may be, for example, once every two days, once a day, twice a day, three times a day, or four times a day. In some embodiments of the present disclosure, the administration of 7-DHC may be administered three times per week.

In some embodiments of the present disclosure, the 7-DHC or a salt or a derivative thereof are administered subcutaneously, intravenously, intradermally, intraperitoneally, orally, intramuscularly or intracranially to the subject.

In some embodiments of the present disclosure, the 7-DHC or a salt or a derivative thereof may be administered to the subject for a period of time sufficient to prevent or treat pulmonary arterial hypertension. In some embodiments of the present disclosure, the sufficient period of time may depend on the species, gender, weight or age of the subject, the stage, symptoms or severity of the disease, as well as the route, time or frequency of administration, etc. In some embodiments of the present disclosure, the administration of 7-DHC or a salt or a derivative thereof is daily for at least 1 month. For example, the administration period of 7-DHC or a salt or a derivative thereof may last 1, 2, 3, 4 or 6 months, or 1, 2, 3 or 4 years, or even longer, as long as no side effects occur during the treatment, and there are no specific limitations in the present disclosure. In the embodiment illustrated in the present disclosure, the period may range from 1 month to 2 years. In other embodiments of the present disclosure, the period may range from 4 weeks to 12 months. In other embodiments of the present disclosure, the 7-DHC or a salt or a derivative thereof are administered daily for a period of at least 2 months.

In at least one embodiment of the present disclosure, the 7-DHC or a salt or a derivative thereof may be administered in an oral dosage form.

In at least one embodiment of the present disclosure, the 7-DHC or a salt or a derivative thereof administered to the subject may be included in a pharmaceutical composition. In at least one embodiment of the present disclosure, the pharmaceutical composition of the present disclosure includes 7-DHC or a salt or a derivative thereof, and pharmaceutically acceptable excipients thereof. In at least one embodiment, the composition of the present disclosure may be formulated in a form suitable for oral administration, such that the composition may be administered to the subject by oral delivery. In other embodiments of the present disclosure, the composition can be formulated into troches, tablets, liquids, powders, granules, dispersants, pills, dripping pills, capsules, ointments, creams, emulsions, gels, patches, injections, inhalants, sprays and suppositories. In some embodiments of the present disclosure, pharmaceutically acceptable excipients include, but are not limited to, fillers, binders, preservatives, disintegrants, lubricants, suspending agents, wetting agents, solvents, surfactant, acid, flavoring agent, polyethylene glycol (PEG), alkylene glycol, sebacic acid, dimethylsulfoxide, alcohol or any combination thereof.

The pharmaceutical composition of the present disclosure may include 7-DHC or a salt or a derivative thereof as a single active ingredient for preventing or treating pulmonary arterial hypertension. In other words, 7-DHC is served as the only active ingredient in the pharmaceutical composition of the present disclosure for preventing or treating pulmonary arterial hypertension. In this embodiment, the present disclosure provides a safe and effective therapy for preventing or treating pulmonary arterial hypertension by using only 7-DHC or a salt or a derivative thereof as the active ingredient.

In other embodiments of the present disclosure, unless the effects of the present disclosure would be inhibited, the compositions may be administered to the subject in combination with other active ingredients. In some embodiments of the present disclosure, the 7-DHC or a salt or a derivative thereof and other active ingredients can be made into a single composition or individual compositions and provided to a subject in need.

In at least one embodiment, the administration of 7-DHC or a salt or a derivative thereof in the method provided by the present disclosure can be combined with any suitable conventional therapy for pulmonary arterial hypertension. In at least one embodiment of the present disclosure, conventional therapies for pulmonary arterial hypertension include, but are not limited to, calcium blockers, anticoagulants, diuretics, cardiotonic agents, endothelin receptor antagonists, phosphodiesterase type 5 inhibitors and prostacyclin analogs.

As used herein, the term “mammal” refers to any animal classified as a mammal, including humans, domestic and farm animals, and zoo, sports, or pet animals, such as dogs, horses, cats, cows, etc., but is not limited thereto. Preferably, the mammal is human.

Multiple examples have been used to illustrate the present disclosure. The examples below should not be taken as a limit to the scope of the present disclosure.

### EXAMPLES

### Experimental Methods and Steps

The principle of cell viability assay (MTT assay) is to utilize MTT ([3-(4,5-cimethylthiazol-2-yl)-2,5-diphenyl-tetrazolium bromide]), a yellow compound, which is a dye that accepts hydrogen ions and can act on cell mitochondria, to generate purple formazan crystals under the action of succinate dehydrogenase (SDH), and the amount of formazan crystals produced is directly proportional to the number of living cells. DMSO was used to dissolve the formazan crystals and O.D. 570 nm was measured, the O.D. value represents the activity of mitochondria, that is, the number of living cells, therefore, MTT assay can be used as an indicator of cell viability (toxicity). After the drug to be tested was added to the cell culture medium and co-cultured, MTT can be used to detect the toxicity of the drug to the cells and determine the drug's action concentration range. The MTT assay experimental procedure includes implanting 1.5×10⁴ test cells into a 48-well plate, culturing them overnight at 37° C. in a culture medium containing 10% FBS and different drug concentrations. The cell culture medium was aspirated, washed with phosphate buffered saline (PBS), then 10% MTT (3-(4,5-dimethylthiazol-2-yl)-2,5-diphenyltetrazolium bromide) solution was added, placed in a 37° C. incubator to react for 4 hours. Next, the MTT solution was aspirated and 100 μl/well of DMSO was added to completely dissolve the MTT purple crystals. After mixing evenly, the absorbance values at wavelength 570 nm were measured.

BrdU (5-bromo-2-deoxyuridine) cell proliferation assay (BrdU assay)

A 48-well plate was used, and 1.5×10⁴ cells were implanted in each well. After 24 hours, the medium was changed to serum-free culture medium and synchronized for 24 hours. A 2-fold concentration of the compound in serum-free culture medium is prepared and the cells were pretreated for 1 hour. Then, the same volume of culture medium containing 20% fetal bovine serum (FBS) was added to dilute the compound. The final concentration was 10% FBS culture medium and 1 times the concentration of the compound. In the experimental group that added TGF-β, the same volume of TGF-β culture medium containing 10 ng/mL was added, with a final concentration of 5 ng/mL TGF-β and 1 times the concentration of the compound, and in the experimental group added with TGF-β, the entire process was conducted in culture medium containing 0.1% serum. Then, BrdU reagent was added about 6 hours later, and the cells were collected the next day for enzyme-linked immunosorbent assay (ELISA) analysis, the compound treatment time was about 24 hours, and the BrdU treatment time was about 18 hours.

### TGF-β Stimulated Pai-1 Luciferase Assay

MLECs-clone32 cell line was used to measure the impact of different compounds on the downstream gene, plasminogenactivator inhibitor 1 (PAI-1) performance stimulated by TGF-β. MLECs-clone32 is an expression construct that is stably transfected for the Mv1Lu cell line, and the expression construct PAI-1 Luciferase contains a truncated (PAI-1) promoter fused to the firefly luciferase reporter gene^{11,12}. The activity stimulated by the truncated PAI-I promoter was quantified by using a luciferase assay. The luciferase activity stimulated by TGF-β in cells without compound treatment was set as 100%, and the effects of each compound on TGF-β-stimulated PAI-I promoter activity were compared. The steps for measuring Pai-1 luciferase activity include using a 96-well plate (DMEM medium containing 10% FBS), seeding each well with 1.5×10⁵ cells, and culturing in an incubator overnight. After discarding the medium the next day, cells in DMEM medium with or without the addition of test compounds were treated with recombinant TGF-β (50 pM) and allowed to react in a 37° C. incubator for 4 hours. The cell culture medium was aspirated, washed with PBS, and the activity of Pai-1 luciferase was evaluated by luciferase assay.

Pulmonary arterial hypertension animal experimental model

The present disclosure utilizes injection of monocrotaline to induce symptoms of pulmonary arterial hypertension in rats to establish a pulmonary arterial hypertension animal experimental model suitable for screening test drugs.

The diagnostic method of clinical pulmonary arterial hypertension standards is right heart catheterization, which is defined as mean pulmonary arterial pressure (mPAP) greater than or equal to 20 mmHg. The diagnostic criteria for pulmonary arterial hypertension, in addition to an increase in mean pulmonary arterial pressure, need to be combined with pulmonary vascular resistance (PVR) greater than 3 Wood unit. The right ventricle pressure (RVP) and systemic mean blood pressure (mbp) in rats with pulmonary arterial hypertension were measured by Millar Model PCU200 pressure system, and the heart rate (HR) was also monitored at the same time. After measuring the hemodynamic data, the arterial blood was extracted from the carotid artery of the pulmonary arterial hypertension rat, and the Abbott blood oximeter (i-STAT) combined with Abbott's special chip cartridge (i-STAT Cartridge G3+) was used to measure arterial gas analysis data such as pH, PaO₂, PaCO₂, HCO₃ and base excess (BE) value in quantitative whole blood samples. The normal pH value is between 7.35 and 7.45, and the pH value indicates whether the experimental animal has the condition of acidemia, alkalemia, acidosis or alkalosis. The normal value of PaO₂ is between 80 and 100 mmHg, which reflects the effectiveness of gas exchange (ventilation/perfusion). The normal value of PaCO₂ is between 35 and 45 (such as 38 and 42) mmHg, which can evaluate the effectiveness of alveolar ventilation. The acid produced by food, tissue decomposition, inflammation and hypoxia rely on the lungs to excrete volatile acid CO2 (short-term) and the bicarbonate (HCO₃⁻) produced by the kidneys (long-term) to neutralize non-volatile acids, while the normal value of bicarbonate hydrogen radical (HCO₃⁻) is between 22 and 26 meq/L. BE is the base excess value, its normal value ranges from −2 to +2, the negative value represents the degree of alkali ion deficiency in the blood, a negative BE value (−) that is too high indicates metabolic acidosis, and a positive value (+) that is too high is metabolic alkalosis. At the end of the experiment, the rats were sacrificed and their hearts were removed and the left and right ventricles were separated and weighed (RV)/(S)+(LV).

### Example 1: Screening TGF-β Receptor Inhibitors by Pulmonary Arterial Hypertension Cell Model

Five compounds to be tested were screened by cell survival assay (MTT assay) and BrdU (5-bromo-2-deoxyuridine) cell proliferation assay, and the information of these compounds was listed in Table 1 below. Tables 2 and 3 respectively listed the growth inhibitions of on human pulmonary artery endothelial cells (hPAEC), human pulmonary artery smooth muscle cells (hPASMC), normal rat pulmonary artery smooth muscle cells (rPASMC, as the control group, represented by “CON”), and rat pulmonary artery smooth muscle cells (represented by “MCT”), by the five compounds to be tested under the conditions of 10% FBS, 0.1% FBS and 100 pM TGF-β. The results showed that the growth inhibition treated with 7-DHC (No. 039) was the most significant.

TABLE 1
Codes, names and structures of the seven compounds

| Code | Name | Structure |
|---|---|---|
| 039 | 7-DHC | 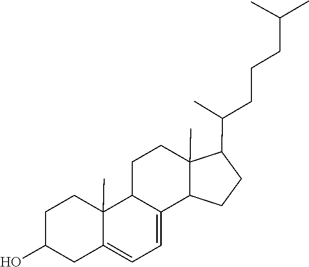 [SMILES: CC(C)CCCC(C)C1CCC2C3=CC=C4CC(O)CCC4(C)C3CCC21C] |
| 042 | Baicalein | 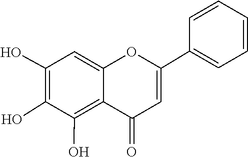 [SMILES: O=c1cc(-c2ccccc2)oc2cc(O)c(O)c(O)c12] |
| 050 | Fisetin | 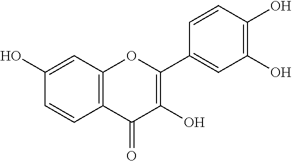 [SMILES: O=c1c(O)c(-c2ccc(O)c(O)c2)oc2cc(O)ccc12] |
| 053 | Macitentan | 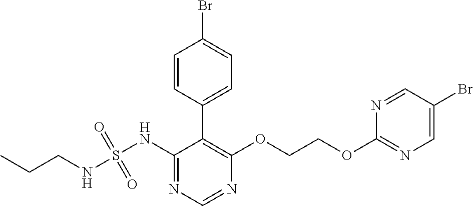 [SMILES: CCCNS(=O)(=O)Nc1ncnc(OCCOc2ncc(Br)cn2)c1-c1ccc(Br)cc1] |
| 056 | Danshensu | 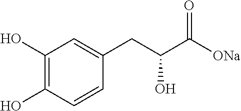 |
| 057 | Melatonin | 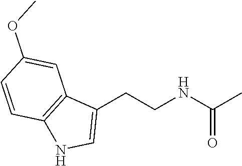 [SMILES: COc1ccc2[nH]cc(CCNC(C)=O)c2c1] |
| 203 | MeTC7 | 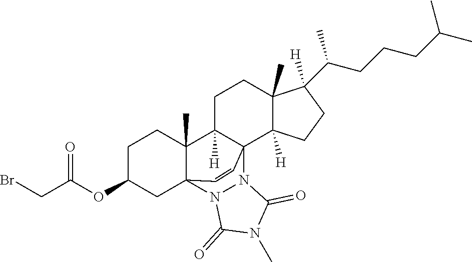 [SMILES: CC(C)CCC[C@@H](C)[C@H]1CC[C@H]2C34C=CC5(C[C@@H](OC(=O)CBr)CC[C@]5(C)[C@H]3CC[C@]12C)n1c(=O)n(C)c(=O)n14] |

TABLE 2
Cell growth inhibitions of the six compounds in 10% FBS

| Code | Cell | 1 μM | 10 μM | 100 μM | EC50 |
|---|---|---|---|---|---|
| 039 | hPAEC | 108% | 76% | 0.3% | 47 μM |
| | hPASMC | 108% | 80% | 12% | 53 μM |
| | CON | 95% | 46% | 2.8% | 38 μM |
| | MCT | 96% | 40% | 3.3% | 37 μM |

| Code | Cell | 5 μM | 25 μM | 50 μM | EC50 | |
|---|---|---|---|---|---|---|
| 042 | hPAEC | 89% | 7% | 4% | 20 | μM |
| | hPASMC | 51% | 36% | 28% | 5 | μM |
| | CON | 16% | 8% | 9% | | |
| | MCT | 19% | 11% | 10% | | |
| 050 | hPAEC | 100% | 14% | 5% | 22 | μM |
| | hPASMC | 90% | 37% | 24% | 27 | μM |
| | CON | 102% | 71% | 68% | 59 | μM |
| | MCT | 84% | 64% | 37% | 38 | μM |
| 053 | hPAEC | 86% | 84% | 77% | 175 | μM |
| | hPASMC | 90% | 82% | 71% | 100 | μM |
| | CON | 101% | 89% | 88% | 142 | μM |
| | MCT | 90% | 79% | 82% | 114 | μM |
| 056 | hPAEC | 89% | 88% | 83% | 272 | μM |
| | hPASMC | 81% | 81% | 71% | 124 | μM |
| | CON | 104% | 88% | 16% | 35 | μM |
| | MCT | 90% | 40% | 15% | 25 | μM |

| Code | Cell | 10 μM | 100 μM | 1000 μM | EC50 |
|---|---|---|---|---|---|
| 057 | hPAEC | 89% | 91% | 81% | |
| | hPASMC | 86% | 89% | 88% | |
| | CON | 99% | 101% | 101% | |
| | MCT | 93% | 93% | 88% | |

*EC50: concentration for 50% of maximal effect

TABLE 3
Cell growth inhibitions of the five compounds
under the addition of 100 pM TGF-β

| No. | Cell | 5 μM | 25 μM | 50 μM | EC50 | |
|---|---|---|---|---|---|---|
| 039 | hPAEC | 111% | 3.5% | 3% | 23 | μM |
| | hPASMC | 95% | 28% | 12% | 24 | μM |
| | CON | 119% | 33% | 25% | 30 | μM |
| | MCT | 87% | 9% | 8.5% | 20 | μM |
| 042 | hPAEC | 70% | 6% | 5% | 15 | μM |
| | hPASMC | 38% | 37% | 33% | 4 | μM |
| | CON | 87% | 65% | 55% | 52 | μM |
| | MCT | 56% | 10% | 10% | 9 | μM |
| 050 | hPAEC | 148% | 105% | 18% | 40 | μM |
| | hPASMC | 119% | 47% | 40% | 35 | μM |
| | CON | 115% | 102% | 86% | 106 | μM |
| | MCT | 142% | 126% | 86% | 79 | μM |
| 053 | hPAEC | 113% | 87% | 29% | 40 | μM |
| | hPASMC | 102% | 78% | 61% | 59 | μM |
| | CON | 102% | 86% | 78% | 96 | μM |
| | MCT | 106% | 94% | 75% | 87 | μM |
| 056 | hPAEC | 121% | 122% | 104% | 155 | μM |
| | hPASMC | 114% | 99% | 84% | 100 | μM |
| | CON | 102% | 94% | 49% | 50 | μM |
| | MCT | 109% | 94% | 12% | 36 | μM |

TABLE 4
IC50 values of the seven compounds inhibiting
TGF-β stimulated Pai-1 luciferase activity (%)

| | | IC50 value of compound inhibiting TGF-β- |
|---|---|---|
| Code | Name | stimulated Pai-1 luciferase activity (%) |
| 039 | 7-DHC | 15.5 μM |
| 042 | Baicalein | 14.8 μM |
| 050 | Fisetin | 44.1 μM |
| 053 | Macitentan | 82.9 μM |
| 056 | Danshensu | N/A |
| 057 | Melatonin | N/A |
| 203 | MeTC7 | 17.5 μM |

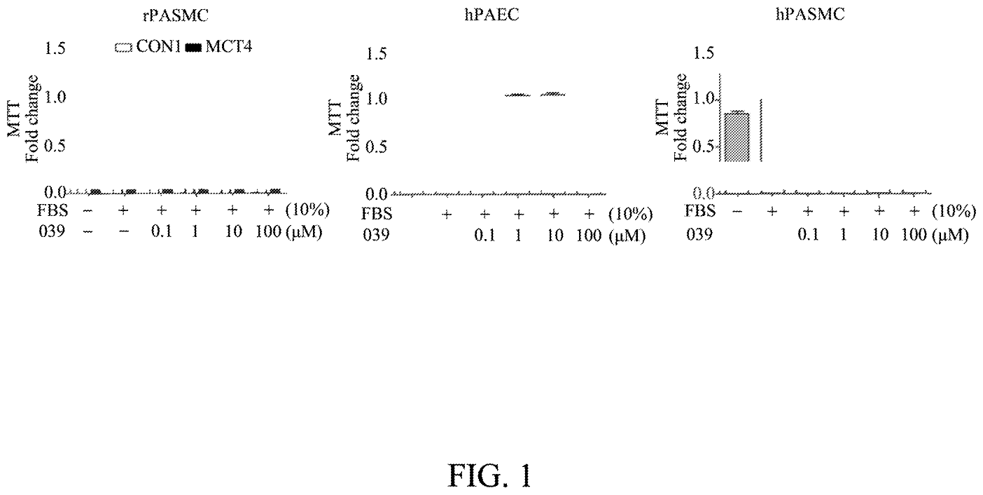

As shown in the results of the cell survival assay, no matter in the normal rat pulmonary artery smooth muscle cells (rPASMC CON1), rat pulmonary artery smooth muscle cells (rPASMC MCT4), human pulmonary artery endothelial cells (hPAEC) and human pulmonary artery smooth muscle cells (hPASMC), when the concentration of 7-DHC (039) was increased to 100 M, cytotoxicity began to occur (FIG. 1).

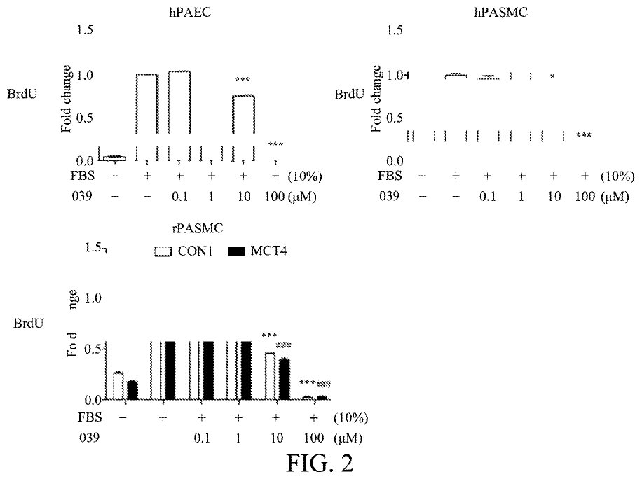

In the BrdU cell proliferation assay, as shown in FIG. 2, under the conditions of 10% FBS, when the concentration of 7-DHC (039) was 10 M, normal rat pulmonary artery smooth muscle cells (rPASMC CON1) and rat pulmonary artery smooth muscle cells (rPASMC MCT4) have significant inhibitory effects, and inhibition of cell growth can also be observed for human pulmonary artery endothelial cells (hPAEC) and human pulmonary artery smooth muscle cells (hPASMC).

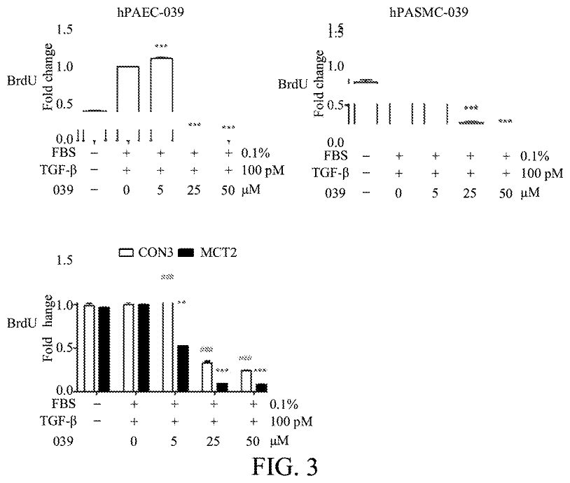

As shown in FIG. 3, under the conditions of 0.1% FBS, 100 pM TGF-β, when the concentration of 7-DHC (039) was 25 μM, the normal rat pulmonary artery smooth muscle cells (rPASMC CON3) and rat pulmonary artery smooth muscle cells (rPASMC MCT2) all had significant inhibitory effects. In particular, the inhibitory effect on rat pulmonary artery smooth muscle cells was more significant. Similarly, when the concentration of 7-DHC (039) was 25 μM, inhibition of cell growth of human pulmonary artery endothelial cells (hPAEC) and human pulmonary artery smooth muscle cells (hPASMC) can also be observed.

### Example 2: Physiological Parameters, Pulmonary Artery Thickening and Arterial gas analysis after pulmonary arterial hypertension animals were administered with 7-DHC

Monocrotaline (MCT) was utilized to induce pulmonary hypertension rat animal model, the TGF-β receptor inhibitor 7-DHC (039) and compound No. 042 were tested via intraperitoneal (IP) administration, and the physiological parameters of the animals were collected, wherein includes measuring right ventricle pressure, systemic arterial pressure, heartbeat and right ventricle hypertrophy, as well as observing and analyzing the pulmonary artery thickening and blood oxygenation levels through pathological sections of lung tissue. Specifically, 7-DHC (039) was divided into two groups with a dosage of 10 mg/kg or 20 mg/kg, the dosage of group 042 was 50 mg/kg, each dosage group has 3 rats (n=3), while the control group (CON) and the pulmonary arterial hypertension rat group (MCT) had 6 rats (n=6) respectively.

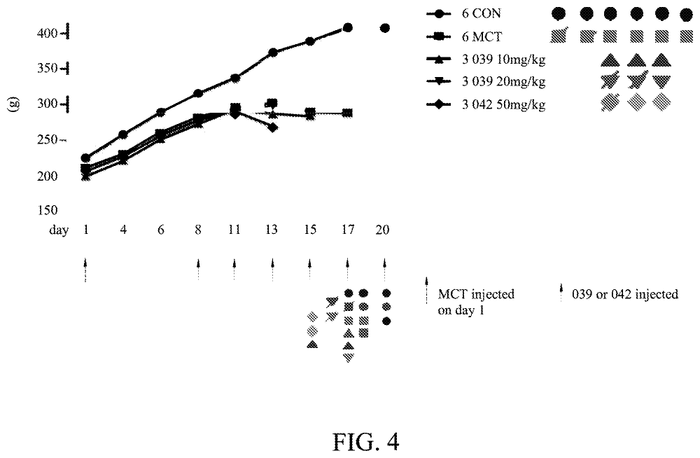

As shown in FIG. 4, the weight and activity of the rats in the 042 administration group had significantly decreased in the second week of administration, so they were sacrificed early on the 21st day after monocrotaline induction. On the 23rd day after monocrotaline induction, it was found that rats in the 20 mg/kg group of 7-DHC (039) had died, therefore, the pulmonary artery pressure could not be obtained, so the right ventricle pressure (RVP) measurement was started on the 24th day.

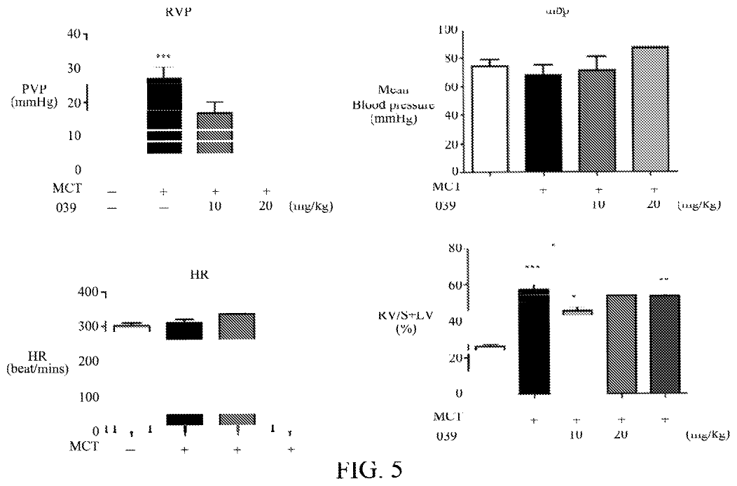

As shown in FIG. 5, the average right ventricle pressure (RVP) of pulmonary arterial hypertension rats (MCT) was 28 mmHg, but right ventricle pressure can be reduced after administration of 7-DHC (039) (10 or 20 mg/kg). After the rats were sacrificed, the hearts were removed and the left and right ventricles were separated and weighed (RV/S+LV), the ratio of the left and right ventricles of normal rats was approximately 25%, the average right ventricle hypertrophy ratio in pulmonary arterial hypertension rats (MCT) exceeds 50%. However, after administration of 7-DHC (039) (10 or 20 mg/kg), right ventricle hypertrophy was reduced, and 7-DHC (039) did not reduce systemic blood pressure and heartbeat, this shows that 10 mg/kg 7-DHC (039) has the effect of reducing right ventricle pressure and reducing right ventricle hypertrophy. In addition, the phenomenon of right ventricle hypertrophy in the 042-administered rats was slightly lower than that in the pulmonary hypertension rats (MCT).

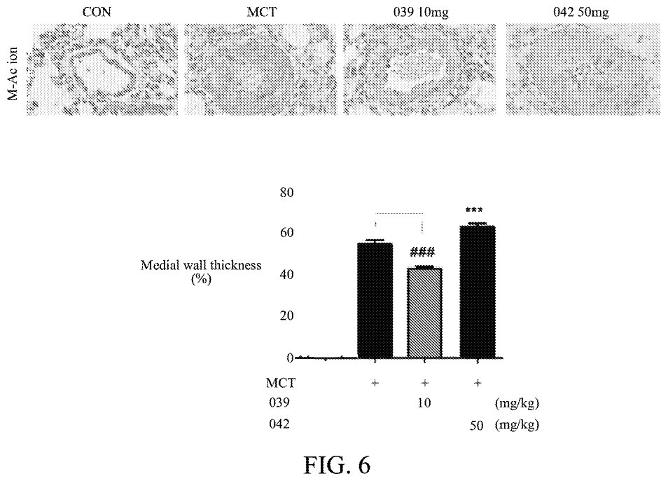

In addition, observe the pulmonary artery tissue sections of the rats in the 042 administration group were observed, and immunohistological staining α-smooth muscle actin (referred to as α-SM actin or α-SMA) was used to confirm the position of the pulmonary artery, the results were shown in FIG. 6, the average pulmonary artery blood vessel thickness (medial wall thickness) of pulmonary arterial hypertension rats (MCT) exceeded 55%. However, in the 10 mg/kg 7-DHC (039) group, the thickness of the pulmonary artery blood vessels was significantly reduced (p<0.001). The above result showed that 10 mg/kg 7-DHC (039) has the effect of reducing the thickness of pulmonary artery blood vessels in rats with pulmonary arterial hypertension.

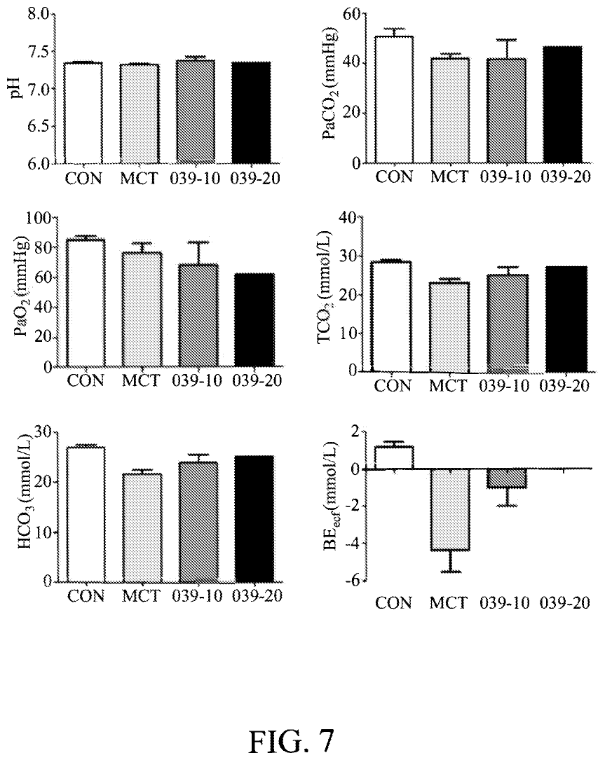

As shown in FIG. 7, the pH value, PaO₂ and PaCO₂ of the control group (CON), pulmonary arterial hypertension rat group (MCT), 10 mg/kg 7-DHC (039) group (represented by “039-10”) and 20 mg/kg 7-DHC (039) group (expressed as “039-20”) did not show any significant differences, and the results may be caused by the longer operation time affecting the blood oxygen exchange function of the lungs. It was interesting that the normal value of base excess (BE) was generally between −2 and +2, however, the BE value in the MCT group was −5.5, indicating metabolic acidosis may be related to tissue hypoxia caused by tissue inflammation or tissue edema. The BE value of the 10 mg/kg 7-DHC (039) group was −2, and the BE value of the 20 mg/kg 7-DHC (039) group was 0. It thus can be seen that 7-DHC (039) can improve tissue inflammation or tissue edema, allowing the tissue to regain oxygen, thereby terminating hypoxic, the lactic acid produced by hypoxia was therefore reduced, and metabolic acidosis was also improved.

Example 3: Comparison of physiological parameters, pulmonary artery thickening and arterial gas analysis values in rats with pulmonary arterial hypertension after administration of 7-DHC or macitentan

Macitentan was a clinical drug currently used for the treatment of pulmonary arterial hypertension, it was a new type of dual endothelin receptor antagonist (ERA) which has high affinity and can occupy the endothelin (ET) receptor on human pulmonary artery smooth muscle for a long time, thereby preventing endothelin-1 (ET-1) from binding to its receptors (ETA and ETB).

Monocrotaline was utilized to induce pulmonary arterial hypertension rat animal model, intraperitoneal injection (IP) TGF-β receptor inhibitor 7-DHC (039) and oral administration of macitentan (soluble in methylcellulose), 7-DHC (039) was divided into two groups according to the dosage: 10 mg/kg or 20 mg/kg, and the dosage of macitentan group was 30 mg/kg.

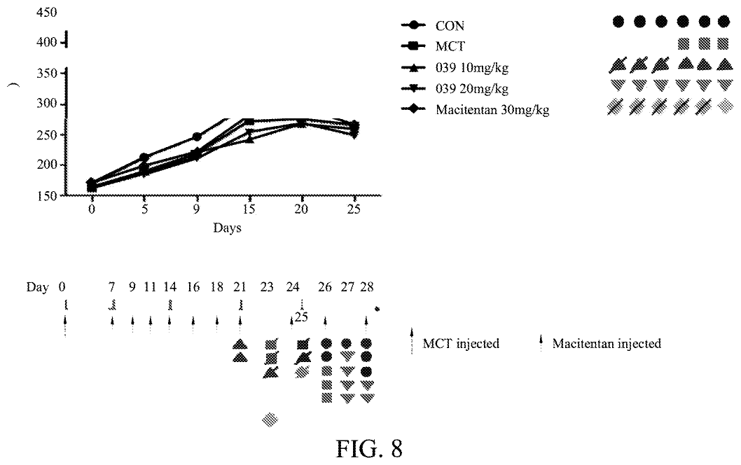

The body weight and activity of the rats in the macitentan group were not greatly affected during the second week of pulmonary arterial hypertension animal model administration, however, on the 23rd day after monocrotaline induction, the rats in the macitentan group died, so the pulmonary artery pressure of this group could not be obtained subsequently. By contrast, although the weight of rats in the 20 mg/kg 7-DHC (039) group decreased significantly, it did not affect their activity, and the survival rate was 100% (FIG. 8).

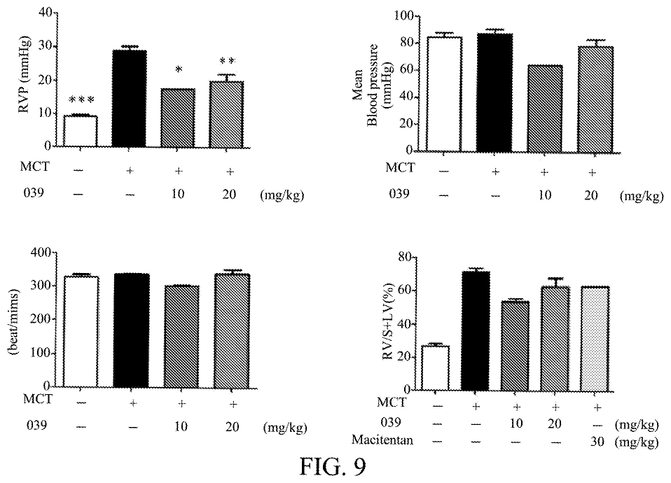

On the 25th day after monocrotaline induction, the right ventricle pressure (RVP) of rats in each group was measured. As shown in FIG. 9, the mean pulmonary artery pressure (RVP) of pulmonary arterial hypertension rats (MCT) was 28.97 mmHg, and the right ventricle pressure of the 10 mg/kg group and 20 mg/kg group administered with 7-DHC (039) was reduced respectively to 17.61 mmHg and 19.96 mmHg. After the rats were sacrificed, the hearts were removed, and the left and right ventricles were separated and weighed (right ventricle/septum+left ventricle; RV/S+LV). The ratio of left and right ventricles in normal rats was approximately 26.7%, the average right ventricle hypertrophy ratio in pulmonary arterial hypertension rats (MCT) was approximately 71.64%, and the average right ventricle hypertrophy ratios in the 10 mg/kg group and 20 mg/kg group of administration of 7-DHC (039) were 53.96% and 62.97%, respectively, both of which significantly reduced the phenomenon of right ventricle hypertrophy. Moreover, 7-DHC (039) did not significantly reduce the systemic average blood pressure and heartbeat, and the administration of 7-DHC (039) having the effect of reducing right ventricle pressure was determined.

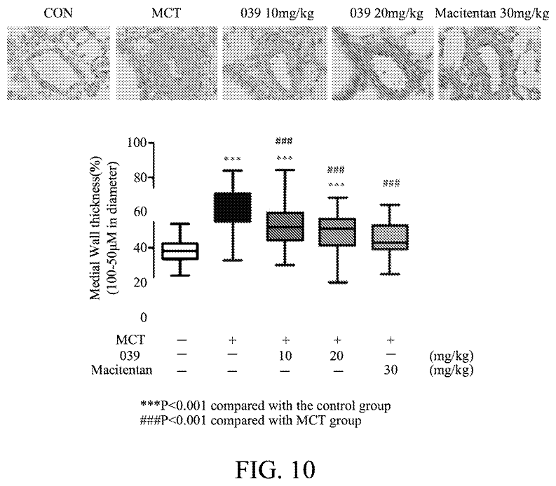

The pulmonary artery vascular tissue sections of rats in each administration group were observed, and the location of the pulmonary artery by immunohistological staining for α-smooth muscle actin were confirmed. As shown in FIG. 10, the results showed that the mean arterial blood vessel thickness of pulmonary arterial hypertensive rats (MCT) exceeded 65%, but the 10 mg/kg group and 20 mg/kg group that were administered 7-DHC (039) significantly reduced the thickening of pulmonary arteries (###p<0.001). Similarly, since macitentan was an endothelin-1 receptor antagonist, a reduction in blood vessel thickening was also observed in the macitentan group, but the survival rate was significantly worse than that of the 7-DHC (039) group. Therefore, the TGF-β receptor inhibitor 7-DHC can inhibit the cell proliferation and remodeling of pulmonary blood vessels by reducing TGF-β-related signaling.

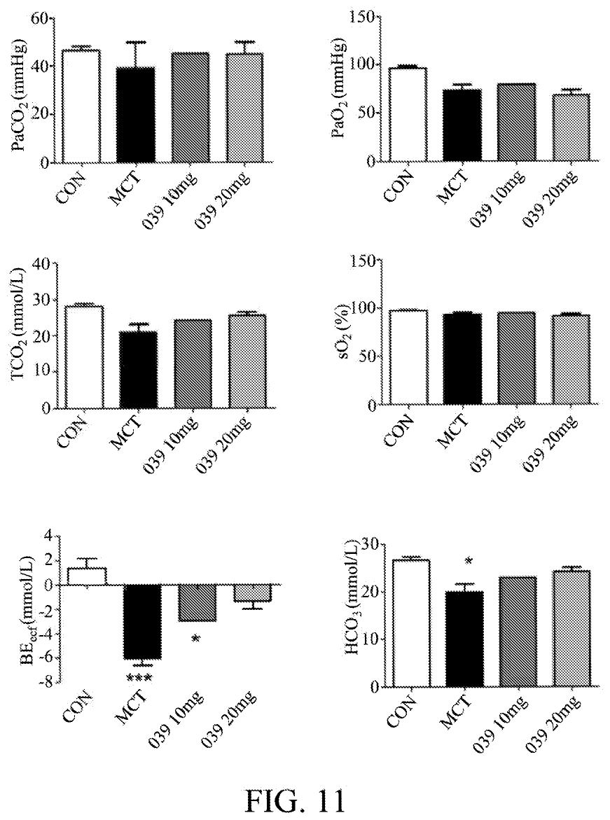

FIG. 11 indicated that there was no significant difference in the PaO₂ and PaCO₂ of the control group (CON), the pulmonary arterial hypertension rat group (MCT), the 10 mg/kg 7-DHC (039) group and the 20 mg/kg 7-DHC (039) group. This result may be caused by the long operation time affects the blood oxygen exchange function of the lungs. The normal value of base excess value (BE) was −2 to +2, the BE value in the MCT group was −6, indicating metabolic acidosis, which may be related to tissue hypoxia caused by tissue inflammation or tissue edema, while the BE value of the 10 mg/kg 7-DHC(039) group was −3, and the BE value of the 20 mg/kg 7-DHC(039) group was −1.4. Therefore, it can be seen that 7-DHC (039) can improve tissue inflammation or tissue edema, allowing the tissue to regain oxygen, thereby terminating hypoxic, the lactic acid produced by hypoxia was reduced, and metabolic acidosis was also improved, the results were consistent with the results of Example 2.

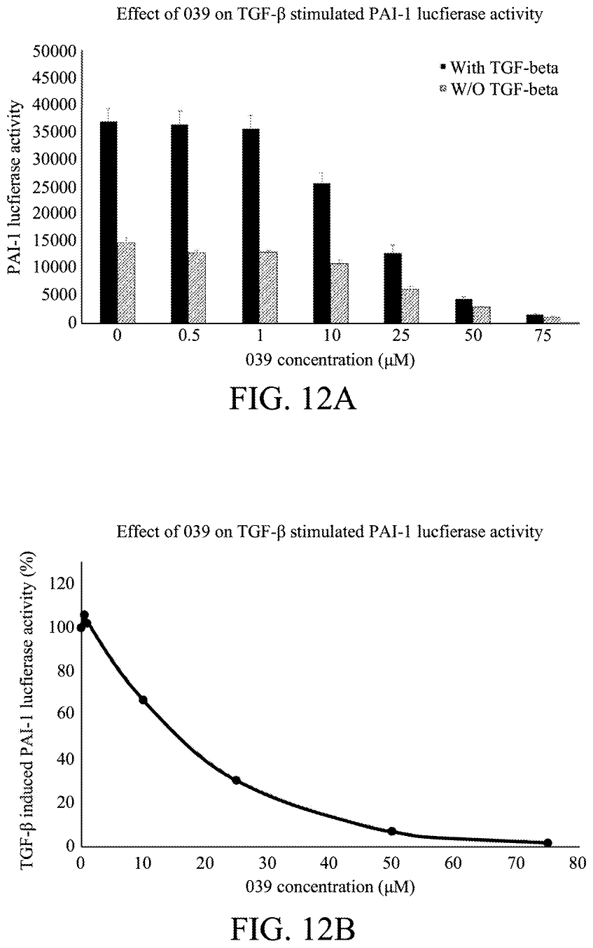

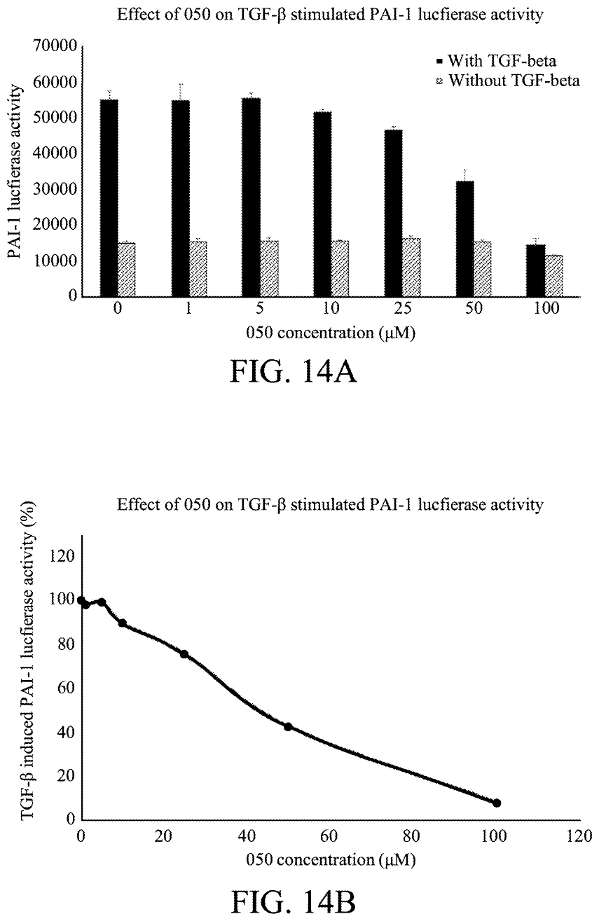

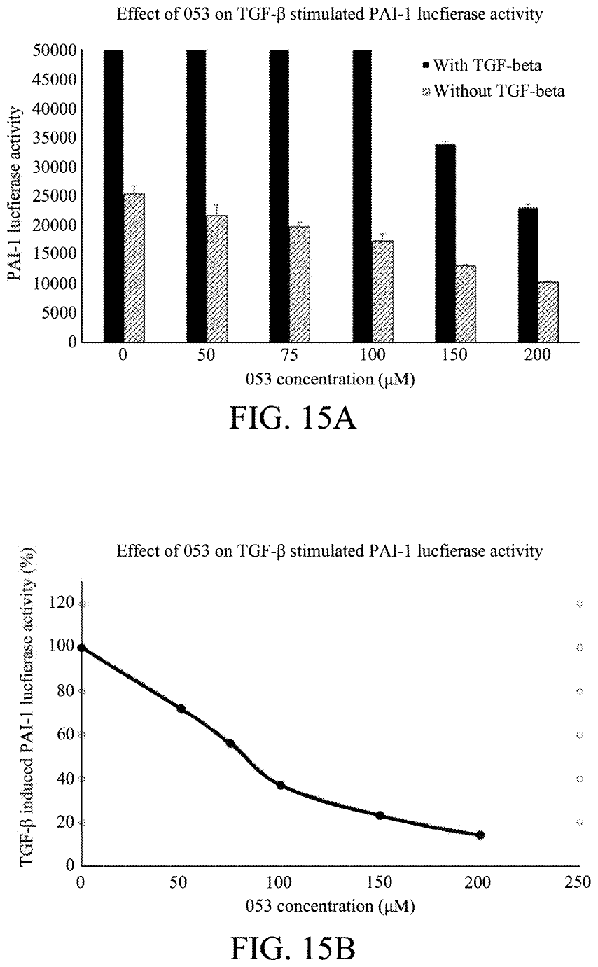

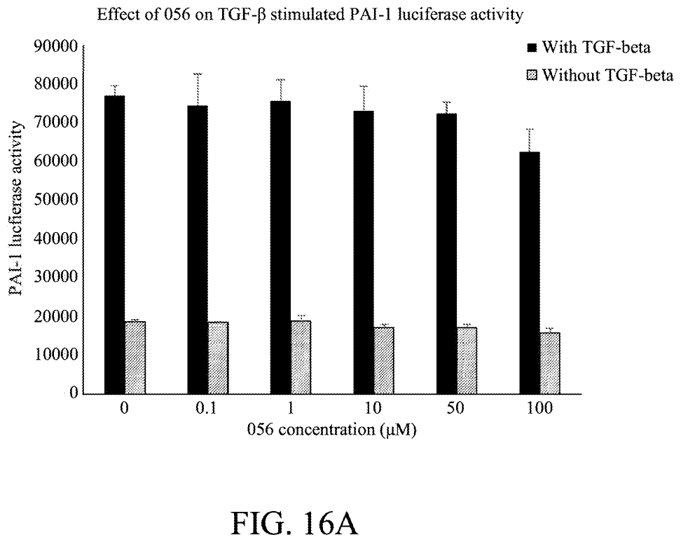

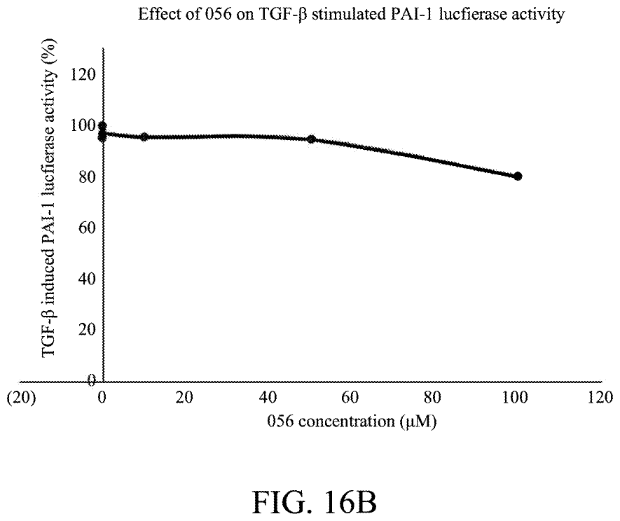

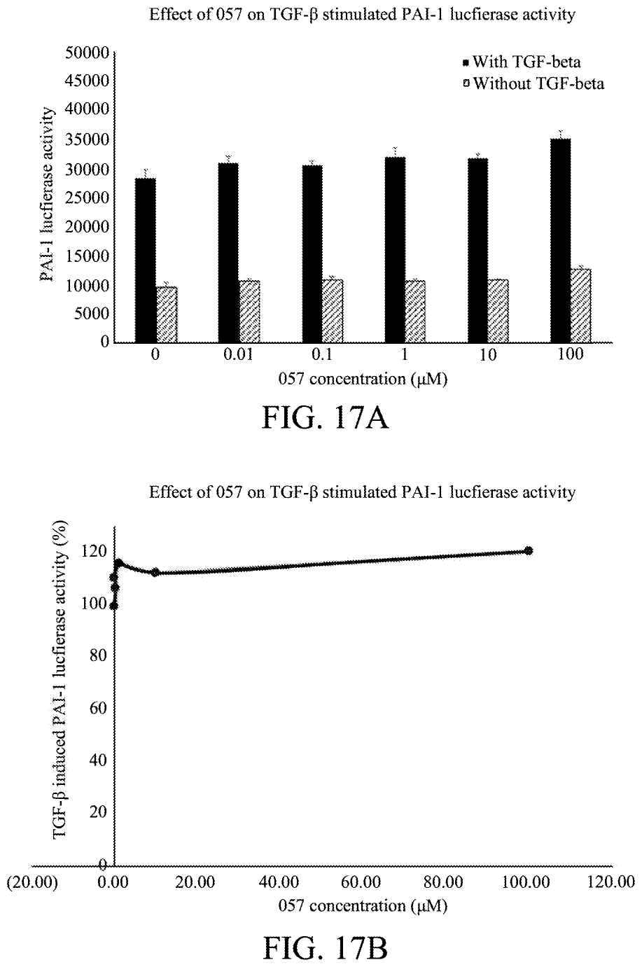

FIGS. 12 to 18 showed the conditions of inhibition of different compounds in different concentration ranges including the conditions of inhibition of Pai-1 luciferase activity with or without TGF-β stimulation by compound 7-DHC (039), Baicalein (042), Fisetin (050), Macitentan (053), Danshensu (056), Melatonin (057) and MeTC7(203), and the conditions of inhibition of TGF-β-stimulated Pai-1 luciferase activity (%) by different compounds in different concentration ranges.

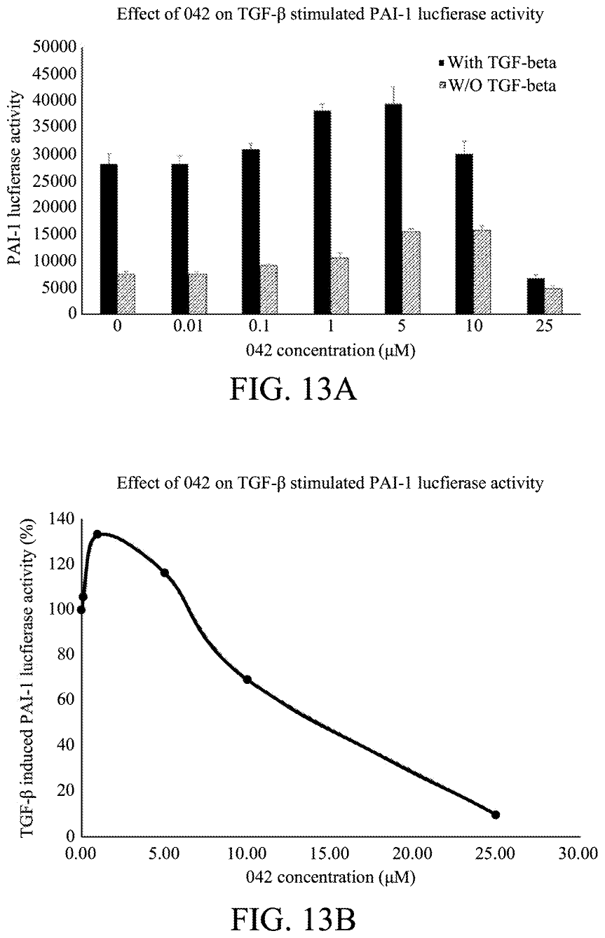

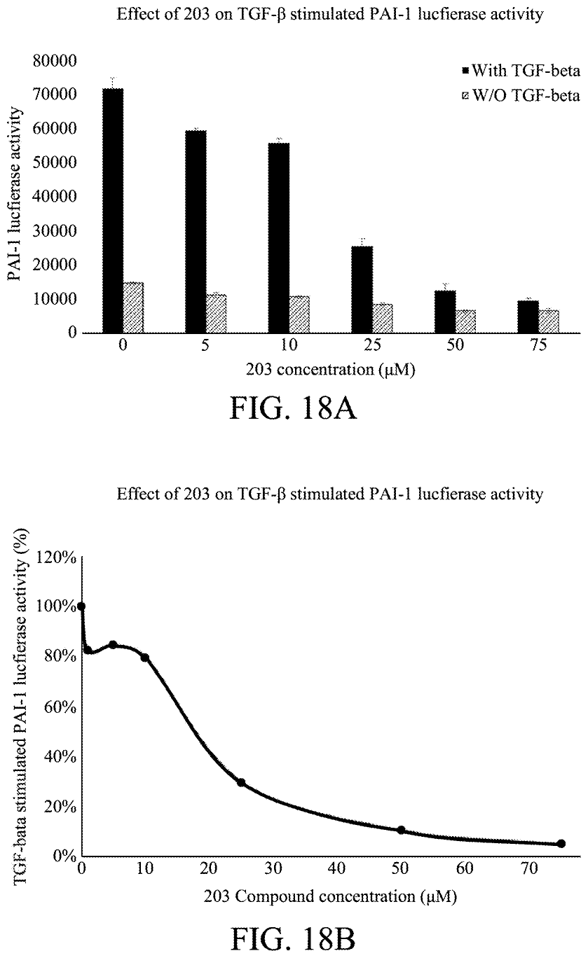

Based on the results of the inhibition of Pai-1 luciferase activity (%) stimulated by TGF-β, it can be found that among the tested compounds 7-DHC, Baicalein, Fisetin, Macitentan, Danshensu, Melatonin or MeTC7, only 7-DHC and 7-DHC derivative MeTC7 could significantly inhibit TGF-β-stimulated Pai-1 luciferase activity when the concentration was increased to M, other compounds failed to have the same effect at similar concentrations. Specifically, for example, as shown in FIGS. 12A and 12B, 7-DHC had a significant inhibitory effect on the activity of Pai-1 luciferase stimulated by TGF-β when the concentration exceeds 10 μM, and the reducing effect increased with concentration. For example, according to FIGS. 13A and 13B, when the concentration of compound Baicalein exceeded 10 μM, a significant inhibitory effect on the activity of Pai-1 luciferase stimulated by TGF-β can be seen, and the reducing effect increased with concentration. For example, as can be seen in FIGS. 18A and 18B, compound MeTC7 had a significant inhibitory effect on the activity of Pai-1 luciferase stimulated by TGF-β when the concentration exceeds 10 μM, and the reducing effect increases with concentration.

In addition, as shown in Table 4, compound 7-DHC, Baicalein, Fisetin, Macitentan, Danshensu, Melatonin, MeTC7 caused TGF-β-stimulated Pai-1 luciferase activity (%) to inhibit the concentration (IC₅₀) value of 50%, the IC₅₀ concentrations of the three compounds 7-DHC, Baicalein, and MeTC7 were lower than those of Macitentan, Danshensu, Fisetin, and Melatonin, and no inhibitory effect was detected. Obviously, compounds 7-DHC, Baicalein and MeTC7 had stronger inhibitory effects on TGF-β-stimulated Pai-1 luciferase activity than the other four compounds.

Even though numerous characteristics and advantages of the present disclosure have been set forth in the foregoing description, together with details of the structure and features of the invention, the invention is illustrative only. Changes may be made in the details, especially in matters of shape, size, and arrangement of parts within the principles of the invention to the full extent indicated by the broad general meaning of the terms in which the appended claims are expressed.

### REFERENCES

1. McGoon M., Gutterman D., Steen V., Barst R., McCrory D. C., Fortin T. A., et al., “Screening, early detection, and diagnosis of pulmonary arterial hypertension: ACCP evidence-based clinical practice guidelines.” CHEST Journal. 2004; 126 (suppl.): 14S-34S.
2. Galie N., Humbert M., Vachiery J. L., et al. “2015 ESC/ERS Guidelines for the diagnosis and treatment of pulmonary hypertension: the Joint Task Force for the Diagnosis and Treatment of Pulmonary Hypertension of the European Society of Cardiology (ESC) and the European Respiratory Society (ERS)”; endorsed by: Association for European Paediatric and Congenital Cardiology (AEPC), International Society for Heart and Lung Transplantation (ISHLT). Eur. Heart J. 2016; 37: 67-119.
3. Simonneau G., Gatzoulis M. A., Adatia I., Celermajer D., Denton C., Ghofrani A., et al., “Updated clinical classification of pulmonary hypertension.” Journal of the American College of Cardiology. 2013; 62 (25): D34-D41.
4. Humbert M., Morrell N. W., Archer S. L., Stenmark K. R., MacLean M. R., Lang I. M., et al. “Cellular and molecular pathobiology of pulmonary arterial hypertension.” Journal of the American College of Cardiology. 2004; 43 (12s1): S13-S24.
5. Cool C. D., Stewart J. S., Werahera P., Miller G. J., Williams R. L., Voelkel N. F., et al. “Three-dimensional reconstruction of pulmonary arteries in plexiform pulmonary hypertension using cell-specific markers: evidence for a dynamic and heterogeneous process of pulmonary endothelial cell growth.” The American Journal of Pathology. 1999; 155 (2): 411-419.
6. Jonigk D., Golpon H., Bockmeyer C. L., Maegel L., Hoeper M. M., Gottlieb J., et al. “Plexiform lesions in pulmonary arterial hypertension: composition, architecture, and microenvironment.” The American Journal of Pathology. 2011; 179 (1): 167-179.
7. Aldred M. A., Vijayakrishnan J., James V., Soubrier F., Gomez-Sanchez M. A., Martensson G., et al. “BMPR2 gene rearrangements account for a significant proportion of mutations in familial and idiopathic pulmonary arterial hypertension.” Human Mutation. 2006; 27 (2): 212-213.
8. Cogan J. D., Vnencak-Jones C. L., Phillips J. A., Lane K. B., Wheeler L. A., Robbins I. M., et al. “Gross BMPR2 gene rearrangements constitute a new cause for primary pulmonary hypertension.” Genetics in Medicine. 2005; 7 (3): 169-174.
9. Cogan J. D., Pauciulo M. W., Batchman A. P., Prince M. A., Robbins I. M., Hedges L. K., et al. “High frequency of BMPR2 exonic deletions/duplications in familial pulmonary arterial hypertension.” American Journal of Respiratory and Critical Care Medicine. 2006; 174 (5): 590-598.
10. Thomson J. R., Machado R. D., Pauciulo M. W., Morgan N. V., Humbert M., Elliott G. C., et al. “Sporadic primary pulmonary hypertension is associated with germline mutations of the gene encoding BMPR-II, a receptor member of the TGF-β family.” Journal of Medical Genetics. 2000; 37 (10): 741-745.
11. An assay for transforming growth factor-beta using cells transfected with a plasminogen activator inhibitor-1 promoter-luciferase construct. Anal Biochem 1994, 216:276-284.
12. Quantification of active and total transforming growth factor-β levels in serum and solid organ tissues. BMC Research Notes 2012, 5:636

What is claimed is:

1. A method for preventing or treating pulmonary arterial hypertension in a subject in need thereof, comprising administering to the subject a pharmaceutical composition comprising an effective amount of a TGF-β receptor inhibitor and a pharmaceutically acceptable excipient thereof.

2. The method of claim 1, wherein the TGF-β receptor inhibitor is 7-dehydrocholesterol, a salt, an oxide, a metabolite or a derivative thereof.

3. The method of claim 2, wherein the 7-dehydrocholesterol salt comprises a acetate salt or a benzoate salt of (3β)-7-dehydrocholesterol or a derivative thereof.

4. The method of claim 2, wherein the 7-dehydrocholesterol derivative comprises cholecalciferol or a compound represented by formula (I): 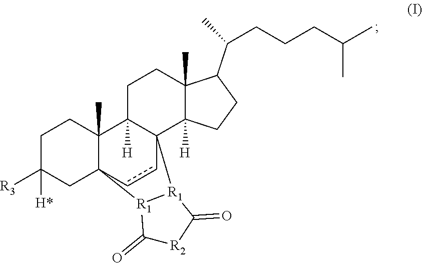
wherein R₁ is CR₅ or N; R₃ is selected from the group consisting of —O(CR₅)ₙR₆, —OC(—O)(CR₅)ₙR₆, —OC(═O)(CR₅)ₙOR₅ and —OC(═O)C(R₅)═C(R₅)₂; R₂ is selected from the group consisting of oxygen, sulfur, C(R₄)₂, and N(R₄); R₄ is independently selected for each occurrence from the group consisting of hydrogen, alkyl, substituted alkyl, cycloalkyl, substituted cycloalkyl, alkenyl, substituted alkenyl, alkynyl, substituted alkynyl, aryl, substituted aryl, aralkyl, substituted aralkyl, heteroaryl, substituted heteroaryl, heteroarylalkyl, substituted heteroarylalkyl, OR₅ and N(R₅)₂; R₅ is independently selected for each occurrence from the group consisting of hydrogen, alkyl, substituted alkyl, cycloalkyl, substituted cycloalkyl, alkenyl, substituted alkenyl, alkynyl, substituted alkynyl, aryl, substituted aryl, aralkyl, substituted aralkyl, heteroaryl, substituted heteroaryl, heteroarylalkyl and substituted heteroarylalkyl; R₆ is selected from the group consisting of fluorine, chlorine, bromine, iodine, methanesulfonyl, toluenesulfonyl, —OSi(R₅)₃, —C(═O)OR₅ and —C(═O)R₅; the dashed lines are single or double bonds; and, n is an integer from 1 to 10.

5. The method of claim 4, wherein the compound represented by formula (I) is the compound represented by formula (Ia) or a salt or a solvate thereof, 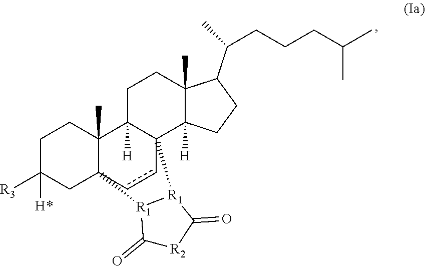
wherein R₁ to R₃ are as defined in claim 4.

6. The method of claim 5, wherein the compound represented by formula (I) is the compound represented by formula (Ib) or a salt or a solvate thereof, 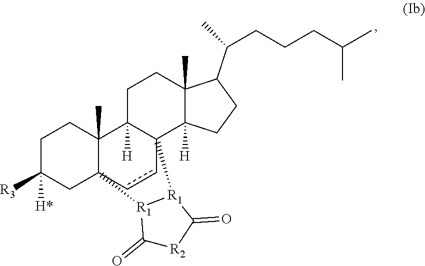
wherein R₁ to R₃ are as defined in claim 4.

7. The method of claim 4, wherein R₁ is N.

8. The method of claim 7, wherein R₂ is N(R₄), and R₄ is as defined in claim 4.

9. The method of claim 8, wherein the compound represented by formula (I) is the compound represented by formula (Ic) or a salt or a solvate thereof, 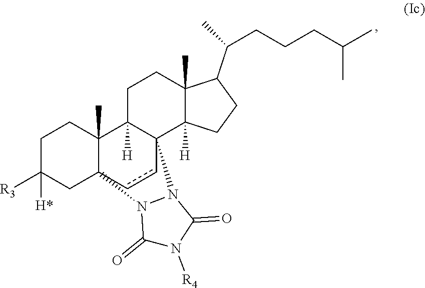
wherein R₃ and R₄ are as defined in claim 4.

10. The method of claim 9, wherein the compound represented by formula (I) is the compound represented by formula (Id) or a salt or a solvate thereof, 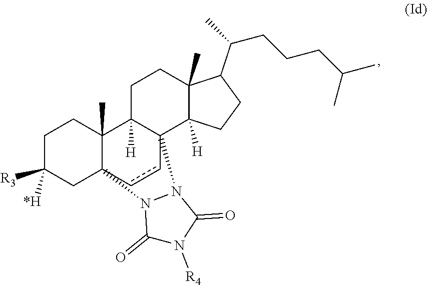
wherein R₃ and R₄ are as defined in claim 4.

11. The method of claim 4, wherein the compound represented by formula (I) is

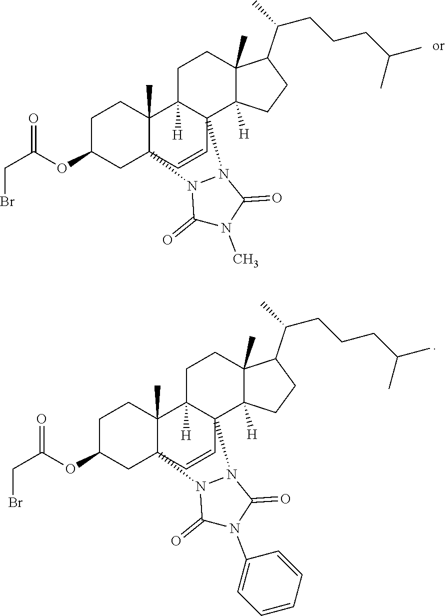

12. The method of claim 4, wherein the subject is a mammal.

13. The method of claim 4, wherein the compound represented by formula (I) is:

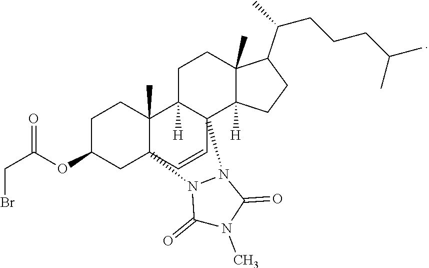

14. The method of claim 2, wherein the 7-dehydrocholesterol, or a salt, an oxide, a metabolite or a derivative thereof is administered to the subject at a dose ranging from 1 mg/kg to 50 mg/kg.

15. The method of claim 1, wherein the 7-dehydrocholesterol, or a salt, an oxide, a metabolite or a derivative is the single active ingredient in the pharmaceutical composition for preventing or treating the pulmonary arterial hypertension.

16. The method of claim 1, wherein the pharmaceutical composition further comprises other active ingredients selected from the group consisting of calcium blockers, anticoagulants, diuretics, cardiotonic agents, endothelin receptor antagonists, phosphodiesterase type 5 inhibitors, prostacyclin analogs, and any combination thereof.

17. The method of claim 1, wherein the TGF-β receptor inhibitor reduces the proliferation of pulmonary artery endothelial cells, smooth muscle cells, or a combination thereof in the subject.

18. The method of claim 1, wherein the pharmaceutical composition is administered to the subject subcutaneously, intravenously, intradermally, intraperitoneally, orally, intrabuccally, sublingually, intramuscularly, into the respiratory tract, or into the lungs.

19. The method of claim 1, wherein the pharmaceutical composition is administered to the subject at least once daily.

20. The method of claim 1, wherein the pharmaceutical composition is administered to the subject for at least 1 month.
